(19) United States
(12) Patent Application Publication
(10) Pub. No.: US 2022/0160554 A1
(43) Pub. Date: May 26, 2022

(54) Baby Blowout Stopper

(71) Applicant: Odette Murat, Patterson, NY (US)

(72) Inventor: Odette Murat, Patterson, NY (US)

(21) Appl. No.: 17/103,864

(22) Filed: Nov. 24, 2020

(51) Int. Cl. A61F 13/84; A61F 13/49

(52) U.S. Cl. CPC A61F 13/84; A61F 13/49

(57) ABSTRACT

An add-on baby blowout stopper for use in a conventional disposable baby diaper made as a slip opened on each side that closes with fasteners and is to receive urine and poop. The Blowout Stopper has an upper side extension made with a fold-over in which a pocket is formed. The fold-over extension and the pocket combined to contain baby blowouts within the conventional disposable diaper and the blowout stopper.

### TECHNICAL FIELD

The present disclosure relates generally to the protection of baby blowouts. More particularly, the present disclosure relates to a separate disposable and attachable add-on to regular diapers to prevent the spill of baby blowouts beyond the top of the diaper area, soiling a baby's upper back area, clothing, crib and all other locations a baby can be at the time of the blowout.

### BACKGROUND

Current day disposable diapers are typically thin and made with materials that allow for the absorption of urine which occurs gradually. As a baby urinates, the material increases in volume. The diaper becomes saturated over a period of time, and a “wetness indicator” informs parents and caregivers that the diaper must now be changed. However, the aforementioned materials do not perform in the same manner when diaper conditions are watery fecal matter which is distinctly thicker than urine.

Baby blowouts are most problematic when it occurs while a baby is lying on his or her back in which case the blowout shoots up the baby's back, onto clothing and bed covers. It can happen on an outing in which case it soils the car seat and clothing or the people holding the baby.

The universal disposable diaper is used as the basic protection to keep babies dry and clean from birth until it is no longer a necessity. It is widely used by parents and caregivers, with the exception of those who utilize cloth diapers. The Baby Blowout Stopper is an add-on attached to the inside of the regular disposable diaper with an adhesive mechanism. It is structured to add height and a pocket to receive blowouts and to stop them from spilling beyond the diaper area and other environments wherever the baby is when they occur.

Accordingly, one major limitation of the current disposable diaper structure is its inability to capture and contain the poop.

Various other diaper accessories have been made in an attempt to stop blowouts. While these accessories may be suitable for the particular purpose employed, they would not be sufficiently efficient for the purpose of the present disclosure as disclosed hereafter.

In the present disclosure, where document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to effectively stop baby blowouts from soiling the baby's upper back and environment when poop shoot up pass the current designed disposable diapers. Accordingly, an add-on-to-diaper accessory provides the necessary protection to contain blowouts within the diaper and blowout stopper areas.

An-other aspect of an example embodiment in the present disclosure is to provide an add-on blowout stopper that works in conjunction with existing conventional disposable diapers. Accordingly, the add-on blowout stopper adheres directly on the conventional disposable diaper.

It is yet another aspect of an example embodiment in the present disclosure to provide an add-on blowout stopper that adheres to the inside of the conventional disposable diaper and is provided with a pocketed extension.

Accordingly, the present disclosure describes an add-on blowout stopper for use inside a conventional baby disposable diaper to stop blowouts from soiling babies environments where they are at the time of the blowouts.

The present disclosure addresses the foregoing disadvantages. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, elements are depicted by reference numerals. The drawings are briefly described as follows.

FIG. 1 is a perspective view, illustrating an embodiment of the add-on Blowout Stopper, per se.

FIG. 2 is a diagrammatic view illustrating the add-on Blowout Stopper's particularities.

FIG. 3 is a diagrammatic perspective view, illustrating the add-on Blowout Stopper affixed to a conventional disposable diaper wherein the add-on Blowout Stopper is apposed and lined up against the back of the conventional disposable diaper.

FIG. 4 is a diagrammatic perspective view, illustrating the add-on Blowout Stopper in use on a disposable diaper being worn by a baby.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

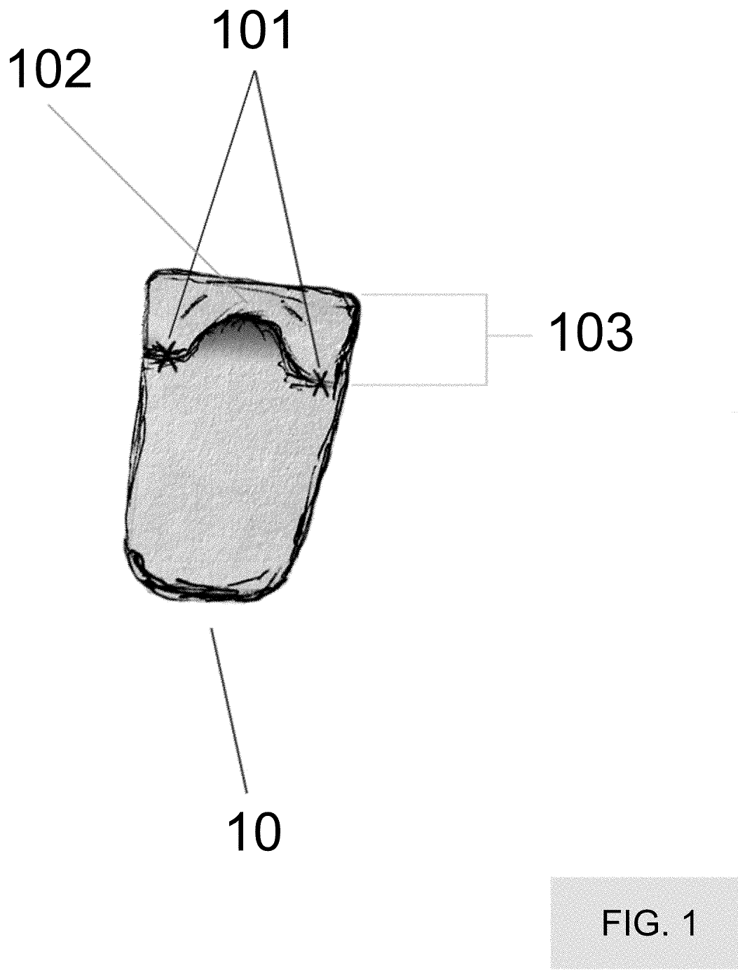

FIG. 1 illustrates an add-on Blowout Stopper 10, two stitches, one on each side, forming two angles 101, a pocket 102 and a fold-over 103.

The Blowout Stopper 10 includes a fold-over 103 from which a pocket is made. The fold-over extends beyond the top of a conventional disposable diaper 70, and combines with the added absorbent material of which it is made to keep baby blowouts from soiling clothing and other items in proximity.

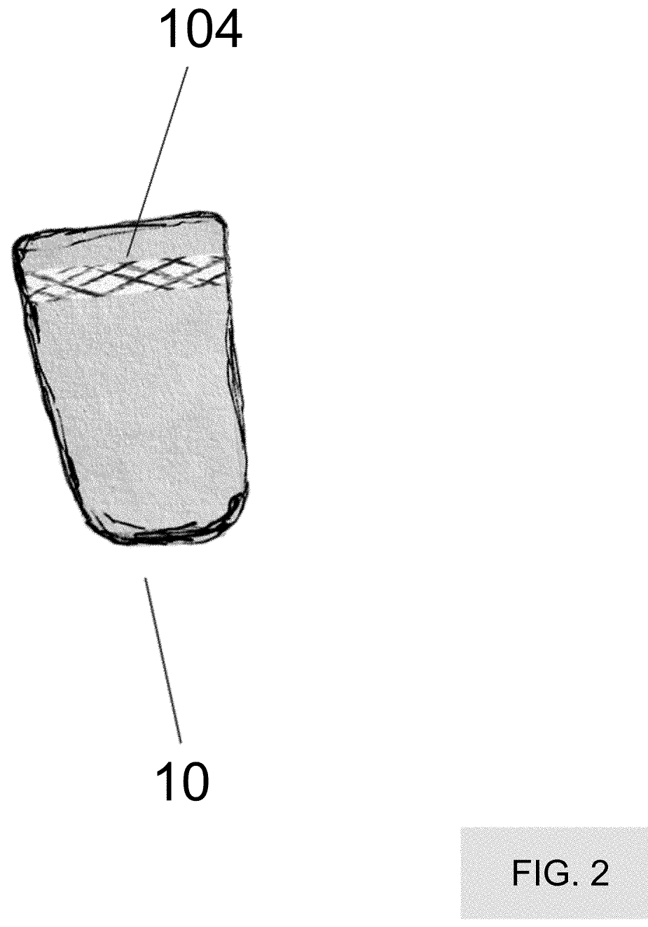

Referring now to FIG. 2, the Blowout Stopper 10 is shown on its back side, depicting an adhering system 104 used to secure it to a conventional disposable diaper 70.

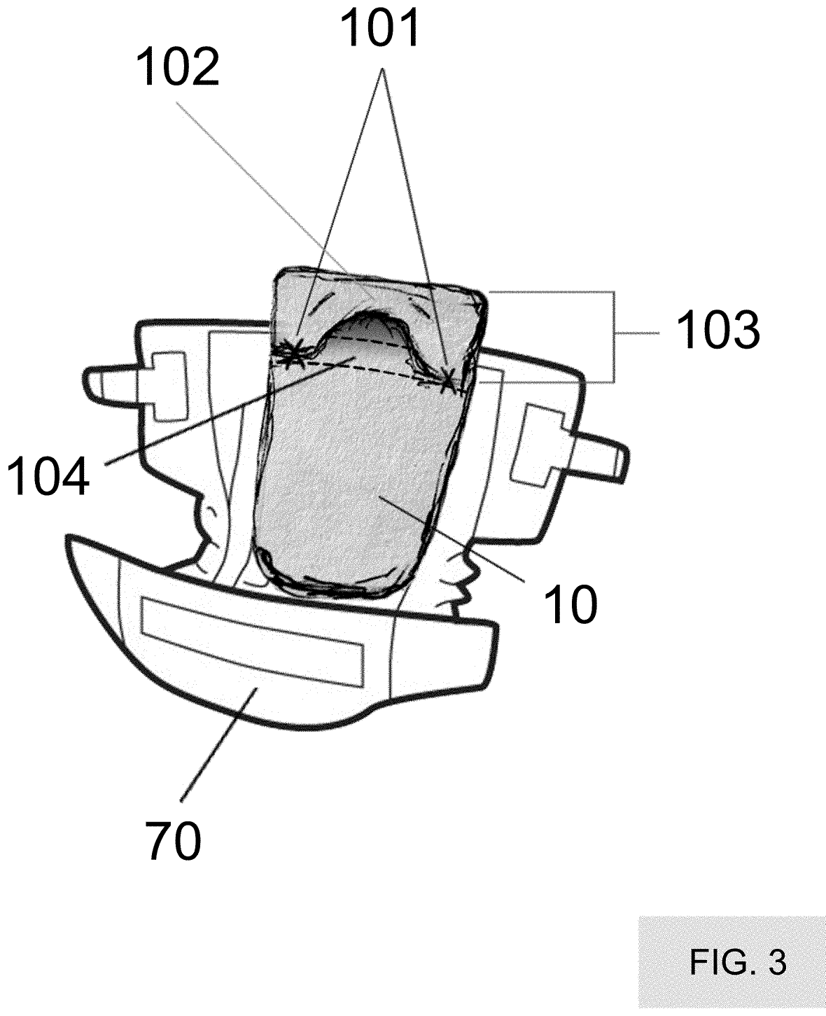

FIG. 3, illustrates the Blowout Stopper 10 affixed to a conventional disposable diaper 70 showing the fold-over extension 103, the pocket 102, the angular formation of the stitches 101 and the location of the adhering mechanism 104.

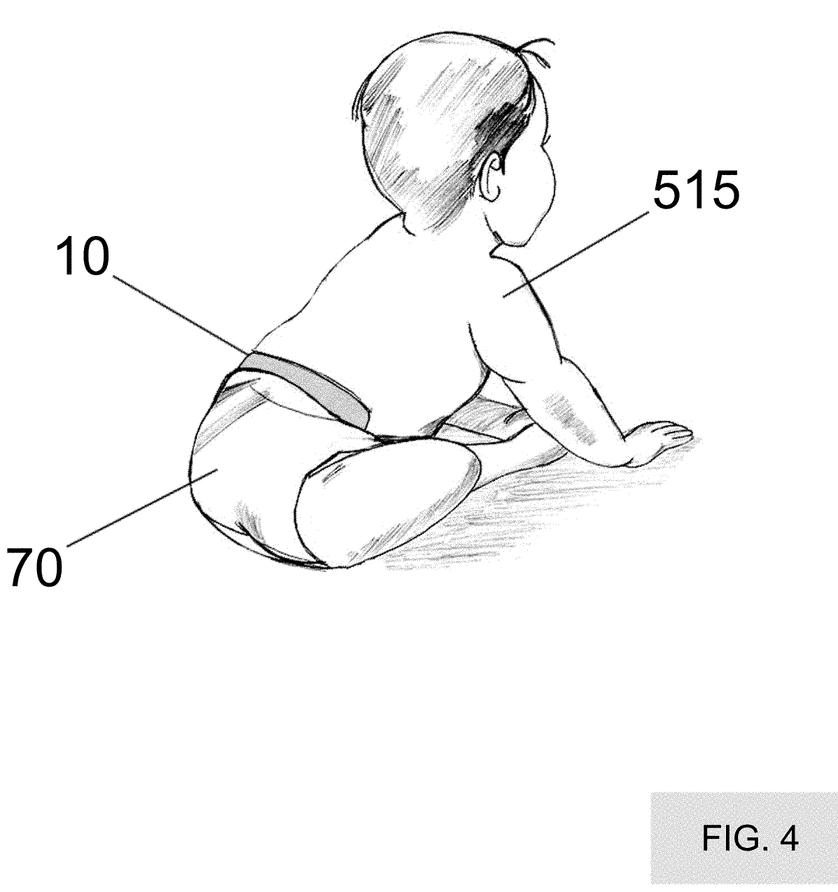

Referring to FIG. 4, the Blowout Stopper 10 is in use on a subject baby 515 with its extended part in full view. The Blowout Stopper 10 is affixed to the conventional disposable diaper 70.

It is understood that when an element is referred hereinabove as being “on” another element, it can be directly on the other element or intervening elements may be present in between.

Moreover, any components or materials can be formed from a same structurally continuous piece or separately fabricated and connected.

Example embodiments are described herein with reference to cross section illustrations that are schematic illustrations of idealized embodiments, As such, variations from the shapes of the illustrations as a result, for example of manufacturing techniques and/or tolerances, are to be expected. Thus, example embodiments described herein should not be construed as limited to the particular shapes of the regions as illustrated herein, but are to include deviations in shapes that result, for example from manufacturing. Thus, the illustrations in the figures are schematic in nature and their shapes are not intended to limit the scope of the present claims.

1. In conclusion, herein is presented an add-on baby blowout stopper. The disclosure is illustrated by example in the drawing figures, and throughout the written description. It should be understood that numerous variations are possible, while adhering to the inventive concept. Such variations are contemplated as being a part of the present disclosure.
